(19) United States
(12) Patent Application Publication
(10) Pub. No.: US 2004/0096916 A1
(43) Pub. Date: May 20, 2004

(54) Renal cell carcinoma tumor markers

(76) Inventors: Roland Kellner, Heppenheim (DE); Kerstin Matzku, Zwingenberg (DE); Barbara Seliger, Goettingen (DE); Rudolf Lichtenfels, Tuebingen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/473,601

(22) PCT Filed: Mar. 28, 2002
(86) PCT No.: PCT/EP02/03503

(30) Foreign Application Priority Data
Apr. 3, 2001 (EP) 01108385.4

(51) Int. Cl. G01N 33/574

(52) U.S. Cl. 435/7.23

(57) ABSTRACT

The present invention relates to tumor markers that can be used for creening, for diagnosis, prognosis and dentification of subtpyes of renal cell carcinoma. The present invention also relates to the use of the identified antigenic proteins in immunoassays and to the use of the tumor markers as immunogens for stimulation of an immune response. The invention further relates to the use ofhte tumor markers for the manufacture of antibodies and antibody fusion proteins directed to the tumor markers.

### FIELD OF THE INVENTION

The present invention relates to tumor markers which can be used for screening, diagnosis, and prognosis of renal cell carcinoma (RCC) and for the identification of subtypes of RCC. The invention further relates to the use of the tumor markers as immunogens for stimulation of an immune response and for the manufacture of antibodies and antibody fusion proteins directed to the tumor markers.

### BACKGROUND OF THE INVENTION

MHC class I-associated peptides are largely derived from the proteolytic degradation of cytosolic proteins. Following initial ubiquitination these proteins are cleaved by the large multicatalytic proteasome complex. Some of the constitutive β-subunits, namely Y, Z, and X, as well as interferon (IFN)-γ-inducible subunits, the low molecular weight protein (LMP) subunits LMP2, MECL1 (LMP10) and LMP7, respectively, form the proteolytic active sites of the proteasome complex. The resulting peptides are transported from the cytosol into the endoplasmatic reticulum (ER) by the transporter associated with antigen processing (TAP), a heterodimeric membrane protein comprised of the TAP1 and TAP2 subunits. The peptides are then loaded onto MHC class I molecules within the ER, involving a multi-step assembly process. Newly synthesized MHC class I heavy chains (HCs) associate with the ER resident chaperone calnexin, then bind β₂-microglobulin (β₂-m) and are subsequently incorporated into the large MHC class I peptide loading complex, consisting of the chaperone calreticulin, the thiol oxidoreductase ERp57, the TAP heterodimer and the transmembrane protein tapasin. In addition, heat shock proteins located in the cytosol as well as in the ER can also bind peptides and play an important role in their stabilization and transport. Stably associated MHC class l/peptide complexes then transit via the trans-Golgi apparatus to the cell surface for presentation to CD8⁺ T cells.

In some diseases such as cancer, autoimmune diseases or cardiovascular disorders peptides of normal or abnormal cellular proteins are presented on the cell surface which can not be found on the cell surface of healthy individuals. Therefore, these peptides and proteins can be used as markers for the identification of such abnormal cells. Furthermore, the detection of antibodies in serum or other body fluids directed to these peptides or proteins can also be used as indicator of risk or as prognostic indicator.

Renal cell carcinoma (RCC) represent approximately 5% of all cancer deaths. At the time of presentation, over 50% of the patients have already developed locally advanced or metastatic disease with 5-year survival rates of less than 20%. Although relative resistant to conventional regimens, RCC are partially susceptible to T cell-based immunotherapy.

Proteome analysis serves as an important tool to study changes in the protein expression and modification pattern in cells cultured under defined conditions, during differentiation steps or during physiological/pathophysiological processes (Pandey et al., Nature 2000, 405, 837; Appella et al., Exs. 2000, 88, 1; Gevaert et al. Electrophoresis 2000, 21, 1145).

Recently, proteomics has been employed for the search of diagnostic, predictive and prognostic parameters in tumors of different origin (Alaiya et al., Electrophoresis 2000, 21, 1210; Unwin et al., Electrophoresis 1999, 20, 3629; Jungblut et al., Electrophoresis 1999, 20, 2100). Such tumor markers (i.e., molecules associated with tumors) might be routinely employed for monitoring the disease of patients and might be further helpful in selecting tumor patients for specifically designed immunotherapeutic treatment strategies.

Several strategies exist to define potential target structures for this therapeutic modality, including 2-D PAGE separation (Sarto et al., Electrophoresis 1997, 18, 599; Sarto, et al., Electrophoresis 1999, 20, 3458), SEREX analysis (Scanlan et al., Int. J. Cancer 1999, 83, 456), cDNA expression cloning (Boon et al. Immunol. Today 1997, 18, 267), and subtractive hybridization procedures (Pitzer et al., J. Cancer Res. Clin. Oncol. 1999, 125, 487).

In WO 99/00671 two-dimensional gel electrophoresis followed by Western Blot analysis with patient derived sera several specific β-tubulin isoforms were identified as tumor markers for neuroblastoma.

In WO 00/20586 novel renal cancer associated antigens have been identified by autologous antibody screening of libraries of nucleic acids expressed in renal cancer cells using antisera from cancer patients which can be used as tumor markers.

However, there is a need for additional tumor markers for development of therapeutics and diagnosis applicable to cancer patients having RCC or other cancers and for methods for the identification of RCC and differentiation of subtypes of RCC.

### SUMMARY OF THE INVENTION

One object of the present invention is therefore to provide new tumor markers.

In more detail the invention relates to the use of at least one protein selected from the group consisting of β-actin, γ-actin, α-tubulin, cytokeratin, cytokeratin 8 (CK 8), cytoskeletal tropomyosin, F-actin capping protein, hsp 27, hsp 60, hsp 70, hsp 90, grp 78 (BIP), gp 96, gluthathione-S-transferase, gluthathione synthetase, superoxide dismutae, thioredoxin peroxidase, PA28α, ubiquitin thiolesterase, triosephosphate isomerase, aldose reductase, enoyl-CoA hydratase, α-enolase, annexin II, IV and V, stathmin, nicotinamide-N-methyltransferase, B23/nucleophosmin and vimentin as tumor marker.

Especially the present invention relates to the use of at least one protein selected from the group consisting of β-actin, γ-actin, α-tubulin, β-tubulin, cytokeratin, CK 8, cytoskeletal tropomyosin, F-actin capping protein, hsp 27, hsp 60, hsp 70, hsp 90, grp 78 (BIP), gp 96, gluthathione-S-transferase, gluthathione synthetase, superoxide dismutase, thioredoxin peroxidase, PA28α, ubiquitin thiolesterase, triosephosphate isomerase, aldose reductase, enoyl-CoA hydratase, α-enolase, annexin II, IV and V, stathmin, nicotinamide-N-methyltransferase, B23/nucleophosmin and vimentin as tumor markers for renal cell carcinoma.

Another object is to provide the use of such tumor markers in immunoassays designed to detect the presence of antibodies which specifically bind to the identified tumor markers in the serum of an individual.

It is an object of the present invention to provide immunoassays to detect the presence of antibodies specific to the tumor markers in the serum of an individual. Such immunoassays can be utilized for screening, for diagnostics and prognosis of the disease. In accordance with the invention, measurement of antibody levels in a sample of an individual can be used for the early diagnosis of RCC or other tumors. Moreover, the monitoring of serum antibody levels can be used prognostically to stage progression of the disease.

A further object of the invention is the use of the tumor markers as immunogens for stimulation of a immune response in a individual against the tumor cells in order to inhibit tumor cell growth or kill tumor cells.

It is an object of the present invention to provide medicaments comprising these tumor markers for stimulation of a immune response against the tumor cells to inhibit tumor cell growth and/or to kill tumor cells.

Furthermore, another aspect of the invention is the use of the tumor markers for the manufacture of antibodies or antibody fusion proteins. Such antibodies or antibody fusion proteins may be used as medicament for tumor cell killing or for the inhibition of tumor growth.

Another object of the present invention is to provide methods and kits for the identification of RCC and differentiation of subtypes of RCC with immunohistochemical methods.

Other objects of the present invention are apparent for a skilled person on the basis of the following detailed description.

### BRIEF DESCRIPTION OF THE DRAWINGS

FIG. 1. Targets detected in the screening window for high molecular weight components (7% T/2.5% C gels)

A section of a colloidal coomassie-stained 2D gel (7% T/2.5% C) representing the spot pattern of a total lysate from approximately 5×10⁶ untreated MZ1257RC cells is shown. The proteins were focussed in the first dimension on a nonlinear Immobiline DryStrip (pH3-10, NL; Amersham Pharmacia Biotech, Freiburg, Germany). Relevant target spots detected by positive immunostaining of blots with patient sera are indicated by arrows. The identities of these target spots were analyzed on corresponding gels by peptide mass fingerprinting and/or partial sequencing.

FIG. 2. Targets detected in the screening window for low molecular weight components (16% T/2.5% C gels)

A colloidal coomassie-stained 2D gel (16% T/2.5% C) representing the spot pattern of a total lysate from approximately 2.5×10⁶ untreated MZ1257RC cells is shown. The proteins were focussed in the first dimension on a nonlinear Immobiline DryStrip (pH3-10, NL; Amersham Pharmacia Biotech, Freiburg, Germany). Relevant target spots detected by positive immunostaining of blots with patient sera are indicated by arrows. The identities of these target spots were analyzed on corresponding gels by peptide mass fingerprinting and/or partial sequencing.

FIG. 3. Targets detected in the screening window for low molecular weight components (16% T/2.5% C gels) following IFN-γ stimulation of the cell line MZ1257RC.

A colloidal coomassie-stained 2-D gel (7% T/2.5% C) representing the spot pattern of a total lysate from approximately 2.5×10⁶ IFN-γ stimulated (48 h) MZ1257RC cells is shown. The proteins were focussed in the first dimension on a nonlinear Immobiline DryStrip (pH3-10, NL; Amersham Pharmacia Biotech, Freiburg, Germany). Relevant target spots detected by positive immunostaining of blots with patient sera are indicated by arrows. The identities of these target spots were analyzed on corresponding gels by peptide mass fingerprinting and/or partial sequencing.

FIG. 4. Immunohistochemical analysis of normal kidney tissue and RCC for CK 8, stathmin and vimentin.

Immunohistochemical stainings (400×, left to right) of normal kidney tissue, RCC of clear cell subtype (G2) and RCC of chromophobic subtype (G2) was performed with anti-CK 8, anti-stathmin and anti-vimentin specific mAbs as described in example 5. Strong positive staining for CK8 in the epithelium of the distal tubule and collecting duct system as well as in RCC cells of clear cell and chromophobic subtype is shown. An intermediate to strong positive cytoplasmic staining of the epithelium for stathmin in the distal tubule system; positive cytoplasmic staining of RCC cells of the clear cell subtype as well as scattered infiltrating inflammatory cells and a negative reaction of RCC cells of chromophobic subtype is demonstrated. A strong positive cytoplasmic staining of interstitial cells in normal kidney tissue and RCC cells of the clear cell type, whereas normal tubulus epithelium is negative for anti-vimentin staining. A weak expression of vimentin in RCC cells of the chromophobic subtype is found.

### DETAILED DESCRIPTION OF THE INVENTION

The objects of the present invention are achieved on the basis of the unexpected finding that β-actin, γ-actin, α-tubulin, β-tubulin, cytokeratin, CK 8, cytoskeletal tropomyosin, F-actin capping protein, hsp 27, hsp 60, hsp 70, hsp 90, grp 78 (BIP), gp 96, gluthathione-S-transferase, gluthathione synthetase, superoxide dismutae, thioredoxin peroxidase, PA28α, ubiquitin thiolesterase, triosephosphate isomerase, aldose reductase, enoyl-CoA hydratase, α-enolase, annexin II, IV and V, stathmin, nicotinamide-N-methyltransferase, B23/nucleophosmin and vimentin, preferably annexin II, IV, V, stathmin, vimentin, and B23/nucleophosmin are substrates for the proteolytic degradation by the large multicatalytic proteasome complex in RCC patients and therefore, that peptides of these proteins are renal cancer associated antigens in such individuals. Therefore, these proteins and fragments thereof can be used as tumor markers.

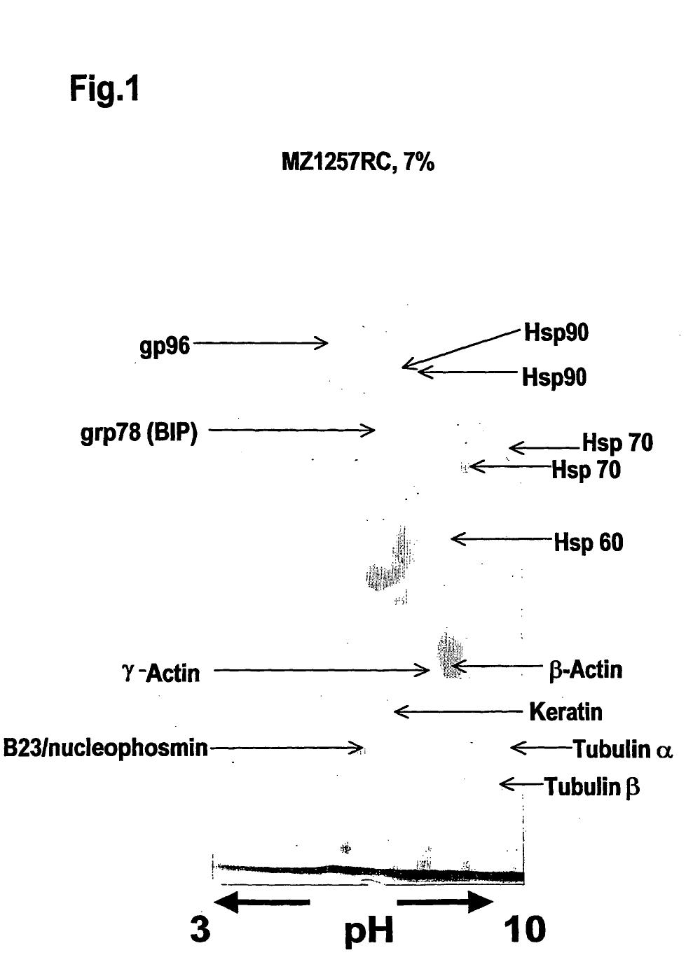

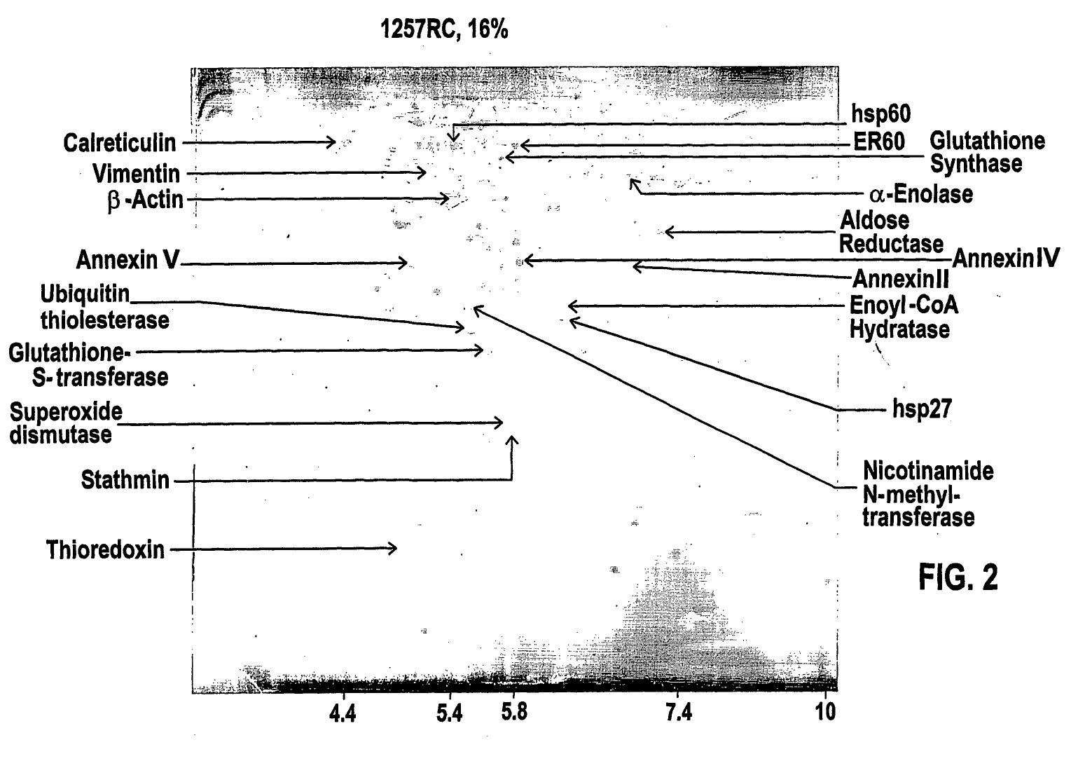

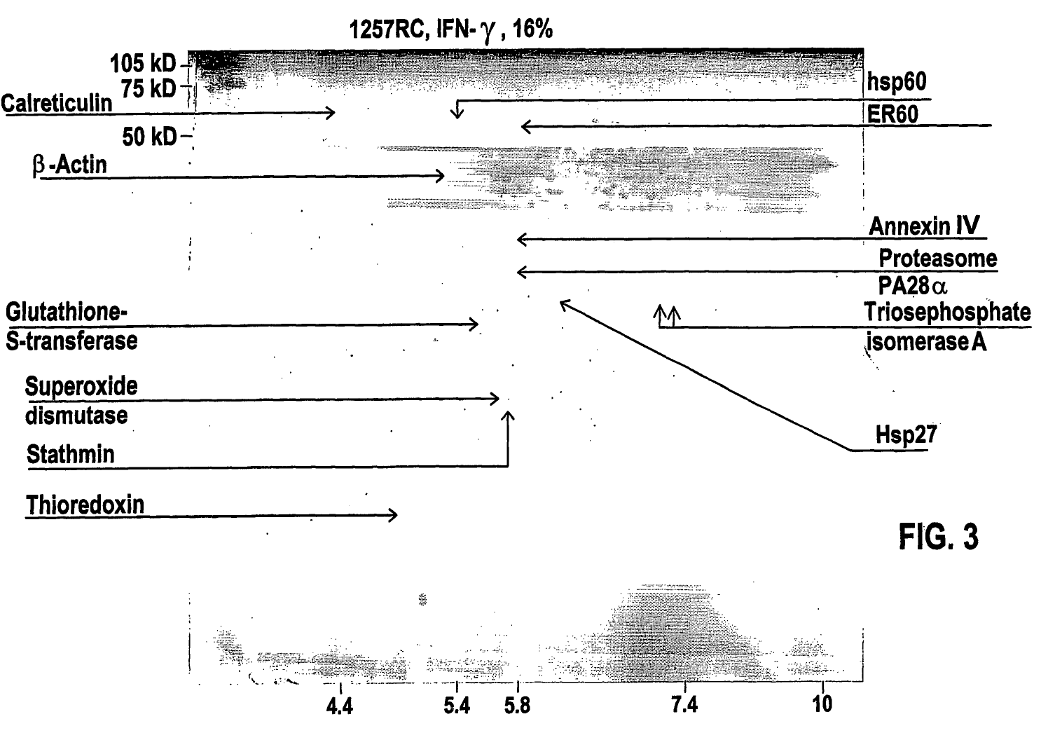

The proteins of the present invention have been identified by two-dimensional gel electrophoresis (see FIGS. 1 to 3) and subsequent detection by immunoblotting with patients' sera. The immunostained protein spots were excised from a duplicate gel, subjected to gel digestion and analyzed by mass spectrometry. With differential analysis of sera from patients versus healthy volunteers the above mentioned proteins were identified as tumor markers in RCC patients.

As used herein, the term “tumor marker” according to the present invention refers to the proteins β-actin, γ-actin, α-tubulin, β-tubulin, cytokeratin, CK 8, cytoskeletal tropomyosin, F-actin capping protein, hsp 27, hsp 60, hsp 70, hsp 90, grp 78 (BIP), gp 96, gluthathione-S-transferase, gluthathione synthetase, superoxide dismutae, thioredoxin peroxidase, PA28a, ubiquitin thiolesterase, triosephosphate isomerase, aldose reductase, enoyl-CoA hydratase, α-enolase, annexin II, IV and V, stathmin, nicotinamide-N-methyltransferase, B23/nucleophosmin and vimentin and immunogenic fragments thereof.

The finding that these proteins are immunogenic in RCC patients provides a basis for development of diagnostic methods for RCC and other cancers in which these proteins are presented at the surface of the tumor cell, as well as a means for monitoring prognosis of various therapeutic treatments for the disease. In addition, this discovery provides a method for use of these proteins as immunogens for stimulation of an immune response against the tumor cells.

Accordingly, the present invention provides the use of the proteins β-actin, γ-actin, α-tubulin, cytokeratin, cytokeratin 8, cytoskeletal tropomyosin, F-actin capping protein, hsp 27, hsp 60, hsp 70, hsp 90, grp 78 (BIP), gp 96, gluthathione-S-transferase, gluthathione synthetase, superoxide dismutae, thioredoxin peroxidase, PA28α, ubiquitin thiolesterase, triosephosphate isomerase, aldose reductase, enoyl-CoA hydratase, α-enolase, annexin II, IV and V, stathmin, nicotinamide-N-methyltransferase, B23/nucleophosmin and vimentin as tumor markers.

Especially, the present invention provides the use of the proteins β-actin, γ-actin, α-tubulin, β-tubulin cytokeratin, CK 8, cytoskeletal tropomyosin, F-actin capping protein, hsp 27, hsp 60, hsp 70, hsp 90, grp 78 (BIP), gp 96, gluthathione-S-transferase, gluthathione synthetase, superoxide dismutae, thioredoxin peroxidase, PA28α, ubiquitin thiolesterase, triosephosphate isomerase, aldose reductase, enoyl-CoA hydratase, α-enolase, annexin II, IV and V, stathmin, nicotinamide-N-methyltransferase B23/nucleophosmin and vimentin as tumor markers for renal cell cancer.

These proteins can be isolated and purified by standard methods including chromatography (e.g., ion exchange, affinity, and size exclusion column chromatography), centrifugation, differential solubility, electrophoresis, or by any standard technique for purification of proteins. The purified proteins can be used in immunoassays designed to detect the presence of antibodies in a sample of an individual, or alternatively, such protein preparations may be used for immunization as described above and below.

The present invention further provides methods for detection and/or quantitative measurement of antibodies directed to the tumor markers of the present invention in a biological sample like serum or other body fluids of patients suffering from RCC or other diseases characterized by the specific presentation of fragments of the tumor markers on the cell surface.

These methods for can be accomplished by any of a number of methods. Such methods include immunoassays which include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays, protein A immunoassays, to name but a few.

For performing the assay, the tumor markers may be immobilized onto a membrane or substrate or may be used in liquid phase. Suitable membarane or substrates are for example a surface capable of binding proteins such as the wells of a polystyrene microtiter plate or a nitrocellulose membrane. Other suitable in vitro assays will be readily apparent to those of skill in the art.

For example, a in-vitro method for diagnosis and prognosis of cancer in an individual, comprising detecting by means of an immunoassay the presence of an antibody obtained from a serum sample of said individual and directed to a tumor marker protein can be carried out by a method comprising the following steps:

a) immobilizing at least one tumor marker onto a membrane or substrate;

b) contacting the membrane or substrate with a serum sample of an individual; and

c) detecting the presence of tumor marker-specific antibodies in the serum sample of the individual.

For the detection of the tumor marker specific-antibodies in the serum sample typically second antibodies labeled with an detectable label are used. The detectable label may be a radioisotope that is detected by autoradiography. Isotopes that are particularly useful for the purpose of the present invention are ³H, ¹²⁵I, ¹³¹I, ³⁵S and ¹⁴C.

The second antibodies may also be labeled with a fluorescent compound. The presence of a fluorescently-labeled antibody is determined by exposing the immunoconjugate to light of the proper wavelength and detecting the resultant fluorescence. Fluorescent labeling compounds include fluorescein, isothiocyanate, rhodamine, phycoerytherin, phycocyanin, allophycocyanin, o-phthaldehyde and fluorescamine.

Alternatively, the second antibody can be detectably labeled by coupling it to a chemiluminescent compound. The presence of the chemiluminescent-tagged immunoconjugate is determined by detecting the presence of luminescence that arises during the course of a chemical reaction. Examples of chemiluminescent labeling compounds include luminol, isoluminol, an aromatic acridinium ester, an imidazole, an acridinium salt and an oxalate ester.

Similarly, a bioluminescent compound can be used to label the second antibody. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds that are useful for labeling include luciferin, luciferase and acquorin.

Alternatively, the second antibody can be detectably labeled by linking the second antibody to an enzyme. When the antibody-enzyme conjugate is incubated in the presence of the appropriate substrate, the enzyme moiety reacts with the substrate to produce a chemical moiety which can be detected, for example, by spectrophotometric, fluorometric or visual means. Examples of enzymes that can be used to detectably label polyspecific immunoconjugates include β-galactosidase, glucose oxidase, peroxidase and alkaline phosphatase. Those of skill in the art will know of other suitable labels which can be employed in accordance with the present invention. The binding of marker moieties to antibodies can be accomplished using standard techniques known to the art.

Typical methodology in this regard is described by Kennedy et al., Clin. Chim. Acta 1976, 70, 1; Schurs et al., Clin. Chim. Acta 1977, 81, 1; Shih et al., Intl. J. Cancer 1990, 46, 1101; Stein et al., Cancer Res. 1990, 50, 1330.

The detection and/or quantitative measurement of antibodies directed to the tumor markers of the present invention in serum or other body fluids can be used in screening of individuals who are at risk for RCC or other disorders characterized by the immunogenic properties of the tumor markers of the present invention. Additionally, measurement of the antibodies may be used prognostically to stage the progression of the disease.

The present invention also provides kits for performing these detection methods. Such a kit can contain all the necessary elements to perform a diagnostic assay described above. A kit will comprise at least one container comprising the tumor marker. The kit may also comprise a second container comprising an antibody or fragment thereof having a appropriate recognition site (for example an anti-human IgG antibody) for the antibodies of the patient serum and a detectable label as described above.

The identification of the tumor markers associated with RCC provides a basis for immunotherapy of the disease. The patient may be immunized with the tumor markers to elicit an immune response which facilitates killing of tumor cells and/or inhibiting tumor cell growth. The tumor markers can be prepared using the methods described above for purification of proteins.

Alternatively, the patient may be treated with antibodies, preferably humanized antibodies or antibody fragments directed to the tumor markers to elicit a reaction which facilitates killing of tumor cells and/or inhibiting tumor cell growth.

The term “antibody fragment” in the meaning of the present invention refers to a portion of an antibody such as F(ab′)₂, F(ab)₂, Fab′, Fab, and the like. Regardless of structure, an antibody fragment binds with the same antigen that is recognized by the intact antibody. The term also includes a synthetic or a genetically engineered polypeptide that binds to a specific antigen, such as polypeptides consisting of the light chain variable region, “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

The term “humanized antibodies” refers to antibodies comprising FRs of the variable regions and constant regions of amino acids located in the light and heavy chain which derive from human sources whereas the hypervariable regions derive from non-human sources.

“FRs” mean the framework regions of an antibody and are found within the variable regions. In these regions a certain alteration of amino acids occurs.

Polyclonal antibodies to the tumor markers of the present invention can be prepared using methods well-known to those of skill in the art. (Green et al., “Production of Polyclonal Antisera,” in: Immunochemical Protocols (Manson, ed.), pages 1-5 (Humana Press 1992); Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995)).

The immunogenicity of the tumor markers can be increased through the use of an adjuvant, such as alum (aluminum hydroxide) or Freund's complete or incomplete adjuvant. Polypeptides useful for immunization also include fusion polypeptides, such as fusions of the tumor marker or a portion thereof with an immunoglobulin polypeptide or with maltose binding protein. The polypeptide immunogen may be a full-length molecule or a portion thereof. If the polypeptide portion is “hapten-like,” such portion may be advantageously joined or linked to a macromolecular carrier (such as keyhole limpet hemocyanin (KLH), bovine serum albumin (BSA) or tetanus toxoid) for immunization.

Monoclonal antibodies to the tumor markers may be obtained by methods known to those skilled in the art (see, for example, Kohler et al., Nature 1975, 256:495 Coligan et al. (eds.), Current Protocols in Immunology, Vol. 1, pages 2.5.1-2.6.7 (John Wiley & Sons 1991); Picksley et al., “Production of monoclonal antibodies against proteins expressed in E coli,” in DNA Cloning 2: Expression Systems, 2nd Edition, Glover et al. (eds.), page 93 (Oxford University Press 1995)). Briefly, monoclonal antibodies can be obtained by injecting mice with a composition comprising one or more of the tumor markers, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

In addition, an anti-tumor marker antibody of the present invention may be derived from a human monoclonal antibody. Human monoclonal antibodies are obtained from transgenic mice that have been engineered to produce specific human antibodies in response to antigenic, challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas.

Methods for obtaining human antibodies from transgenic mice are described, for example, by Green et al., Nature Genet. 1994 7, 13; Lonberg et al., Nature 1994, 368, 856; and Taylor et al., Int. Immun. 1994, 6, 579.

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104 (The Humana Press, Inc. 1992).

For particular uses, it may be desirable to prepare fragments of the anti-tumor marker antibodies. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an Fc fragment directly. These methods are described, for example, by Goldenberg, U.S. Pat. No. 4,331,647, Nisonoff et al., Arch Biochem. Biophys. 1960, 89, 230; Porter, Biochem. J. 1959, 73, 119; Edelman et al., in Methods in Enzymology Vol. 1, page 422 (Academic Press 1967), and by Coligan at pages 2.8.1-2.8.10 and 2.10.-2.10.4.

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody. For example, Fv fragments comprise an association of V_{H} and V_{L} chains. This association can be noncovalent, as described by Inbar et al. Proc. Natl. Acad. Sci. USA 1972, 69, 2659. Alternatively, the variable chains can be linked by an intermolecular disulfide bond or cross-linked by chemicals such as glutaraldehyde (see, for example, Sandhu, Crit. Rev. Biotech. 1992, 12, 437).

The Fv fragments may comprise V_{H} and V_{L} chains which are connected by a peptide linker. These single-chain antigen binding proteins (scFv) are prepared by constructing a structural gene comprising DNA sequences encoding the V_{H} and V_{L} domains which are connected by an oligonucleotide. The structural gene is inserted into an expression vector which is subsequently introduced into a host cell, such as E. coli. The recombinant host cells synthesize a single polypeptide chain with a linker peptide bridging the two V domains. Methods for producing scFvs are described, for example, by Whitlow et al. Methods: A Companion to Methods in Enzymology 1991 2, 97 (also see, Bird et al., Science 1988, 242, 423, Ladner et al., U.S. Pat. No. 4,946,778).

As an illustration, a scFV can be obtained by exposing lymphocytes to the tumor markers in vitro, and selecting antibody display libraries in phage or similar vectors (for instance, through use of immobilized or labeled tumor markers). Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods: A Companion to Methods in Enzymology 1991, 2, 106; Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995), and Ward et al., “Genetic Manipulation and Expression of Antibodies,” in Monoclonal Antibodies: Principles and Applications, Birch et al., (eds.), page 137 (Wiley-Liss, Inc. 1995)).

Alternatively, an anti-tumor marker antibody may be derived from a “humanized” monoclonal antibody. Humanized monoclonal antibodies are produced by transferring mouse complementary determining regions from heavy and light variable chains of the mouse immunoglobulin into a human variable domain. Typical residues of human antibodies are then substituted in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Natl. Acad. Sci. USA 1989, 86, 3833. Techniques for producing humanized monoclonal antibodies are described, for example, by Jones et al., Nature 1986, 321, 522; Carter et al.; Proc. Natl. Acad. Sci. USA 1992, 89, 4285; Sandhu, Crit. Rev. Biotech. 1992, 12, 437; Singer et al., J. Immun. 1993, 150, 2844; Sudhir (ed.), Antibody Engineering Protocols (Humana Press, Inc. 1995); Kelley, “Engineering Therapeutic Antibodies,” in Protein Engineering: Principles and Practice, Cleland et al. (eds.), pages 399434 (John Wiley & Sons, Inc. 1996); and by Queen et al., U.S. Pat. No. 5,693,762 (1997).

Alternatively, the patient may be treated with antibody fusion proteins directed to the tumor marker proteins to elicit a reaction which facilitates killing of tumor cells and/or inhibiting tumor cell growth.

As used herein, the term “antibody fusion protein” refers to a fusion molecule that consists essentially of an antibody or a fragment thereof directed to a tumor marker of the present invention and a therapeutic agent which is fused directly or via a linker or spacer to the immunoglobulin or fragment thereof. Examples of therapeutic agents suitable for such fusion proteins include immunomodulators and toxins, for example but not limited to cytokines such as IL-1, IL-2, IL-4, IL-6, IL-7, IL-10, IL-13, IFNs, TNFα or CSFs.

Fusion proteins can be prepared by methods known to those skilled in the art by preparing each component of the fusion protein and chemically conjugating them. Alternatively, a polynucleotide encoding both components of the fusion protein in the proper reading frame can be generated using known techniques and expressed by the methods described for example in EP0659439.

In one embodiment of the invention an immunogen comprising one or a mixture of the purified tumor markers to which a patient cancer has developed antibodies, is used to elicit an immune response.

In another embodiment of the present invention antibodies or antibody fragments raised against the tumor markers of the present invention may be used to a reaction which facilitates killing of tumor cells and/or inhibiting tumor cell growth. The tumor markers, mixtures thereof or the antibodies and antibody fragments or antibody fusion proteins of the present invention can be applied directly or within pharmaceutical compositions comprising said compounds and a pharmaceutically acceptable diluent, carrier or excipient therefor to patients suffering from RCC or other diseases characterized by the specific presentation of fragments of the tumor markers on the cell surface.

As used herein, the term “pharmaceutically acceptable carrier” means an inert, non toxic solid or liquid filler, diluent or encapsulating material, not reacting adversely with the active compound or with the patient. Suitable, preferably liquid carriers are well known in the art such as sterile water, saline, aqueous dextrose, sugar solutions, ethanol, glycols and oils, including those of petroleum, animal, vegetable, or synthetic origin, for example, peanut oil, soybean oil and mineral oil.

The formulations according to the invention may be administered as unit doses containing conventional non-toxic pharmaceutically acceptable carriers, diluents, adjuvants and vehicles which are typical for parenteral administration.

Many methods may be used to introduce the formulations derived above; including but not limited to oral, intradermal, intramuscular, intraperitoneal, intravenous, and subcutaneous.

The term “parenteral” includes herein subcutaneous, intravenous, intra-articular and intratracheal injection and infusion techniques. Also other administrations such as oral administration and topical application are suitable. Parenteral compositions and combinations are most preferably administered intravenously either in a bolus form or as a constant fusion according to known procedures.

When the compounds of this invention are formulated as a tablet capsule or powder, usual carriers and excipients such as magnesium carbonate, calcium carbonate, sodium bicarbonate, magnesium stearate, calcium stearate, talc, lactose, microcrystalline cellulose, methyl cellulose, sodium carboxymethyl cellulose starch and anhydrous silica, lubricants such as hydrated castor oil, magnesium stearate, sodium lauryl sulfate and sugar, pectin, dextrin, tragacanth, a low-melting wax, cocoa butter, alginates, gelatin, polyvinyl pyrrolidone, polyethyl glycols, quaternary ammonium compounds and the like as well as binders such as starch, glucose, gum arabicum and mannitol can be used. The tablets or capsules may be coated according to methods well known in the art.

Oral liquid preparations may be in the form of aqueous or oily suspensions, solutions, emulsions, syrups or elixirs, or may be presented as a dry product for reconstitution with water or another suitable vehicle before use. Such liquid preparations may contain conventional additives like suspending agents, emulsifying agents, non-aqueous vehicles and preservatives.

Topical applications may be in the form of aqueous or oily suspensions, solutions, emulsions, jellies or preferably emulsion ointments.

In compositions comprising the tumor markers such formulations are preferred wherein the tumor markers are formulated with a suitable adjuvant in order to enhance the immunological response to the protein antigen. Suitable adjuvants include, but are not limited to mineral gels, e.g. aluminum hydroxide, surface active substances such as lysolecithin, pluronic polyols, polyanions, peptides, oil emulsions, and potentially useful human adjuvants such as BCG (bacilli Calmett-Guerin) and (Corynebacterium parvum).

Unit doses according to the invention may contain daily required amounts of the compound according to the invention, or sub-multiples thereof to make up the desired dose. The optimum therapeutically acceptable dosage and dose rate for a given patient (mammals, including humans) depends on a variety of factors, such as the activity of the specific active compound employed, the age, body weight, general health, sex, diet, time and route of administration, rate of clearance, the object of the treatment, i.e., therapy or prophylaxis and the nature of the disease to be treated which are known to the skilled person.

Therefore, in compositions and combinations in a treated patient (in vivo) a pharmaceutical effective daily dose of the active compound of this invention is between about 0.01 and 100 mg/kg body weight, preferably between 0.1 and 10 mg/kg body weight. According to the application form one single dose may contain between 0.01 and 10 mg of the active compound.

The tumor markers, antibodies and antibody fusion proteins of the present invention are also useful in conjunction with other chemotherapeutic agents. Chemotherapeutic agents which may be used in conjunction with the compounds of the present invention includes, according to this invention, agents that exert anti-neoplastic effects, i.e., prevent the development, maturation, or spread of neoplastic cells, directly on the tumor cell, e.g., by cytostatic or cytotoxic effects, and not indirectly through mechanisms such as biological response modification. Chemotherapeutic agents according to the invention are preferably natural or synthetic chemical compounds, but biological molecules, such as proteins, antibodies, chemokines, cytokines, polypeptides etc. are not excluded. There are large numbers of chemotherapeutic agents available in commercial use, in clinical evaluation and in pre-clinical development, which could be included in the present invention.

Examples of chemotherapeutic or agents include alkylating agents, for example, nitrogen mustards, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; cytotoxic antibiotics and camptothecin derivatives. Preferred chemotherapeutic agents or chemotherapy include amifostine (ethyol), cisplatin, dacarbazine (DTIC), dactinomycin, mechlorethamine (nitrogen mustard), streptozocin, cyclophosphamide, carrnustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11,10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil and combinations thereof.

The present invention furthermore provides methods and kits for the identification of RCC and the differentiation of subtypes of RCC with immunohistochemical methods on the basis of the findings described below.

According to the histological features, RCCs are classified into distinct subtypes, the most frequent clear cell, the chromophobic, the chromophilic and the oncocytomic subtype. Methods for the determination of the different subtypes of RCC have been described in Thoenes et al, (Path. Res. Pract. 1986, 181, 125) and Storkel and van der Berg (World J. Urol. 1995, 13, 153).

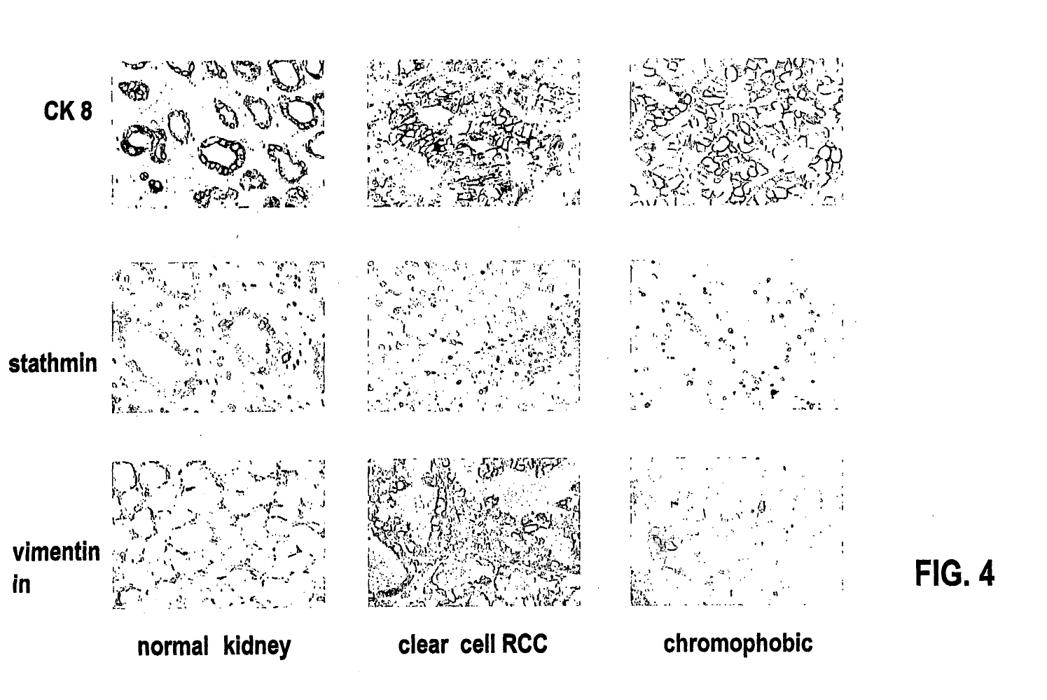

It has been found that the expression pattern of three selective immunogenic proteins differs in normal kidney and distinct subtypes of RCC. The expression pattern of CK 8, stathmin and vimentin, was immunohistochemically analyzed in a series of surgically removed RCC lesions of distinct subtype and autologous normal renal epithelium. As shown in FIG. 4, the epithelium of the proximal and distal tubule system as well as the epithelium of the collecting duct system showed a strong positive staining of the cell membranes for CK 8, whereas all epithelial cells of the normal kidney tissue exhibit negative staining for vimentin. In contrast, the different RCC subtypes showed an intermediate to strong positive staining for CK 8 and vimentin in 36% and 72% of the surgically removed lesions, respectively (FIG. 4, Table 1).

TABLE 1
CK 8 and vimentin expression in different RCC subtypes

| | | | | RCC | | | | | | |
|---|---|---|---|---|---|---|---|---|---|---|
| CK8 | | | | sub- | | | Vimentin | | | |
| +++ | ++ | + | − | type | G | N | +++ | ++ | + | − |
| 3 | 5 | 7 | 2 | clear | 1 | 17 | 11 | 2 | 2 | 2 |
| | | | | cell | | | | | | |
| | | | | type | | | | | | |
| 3 | 1 | 7 | 6 | clear | 2 | 17 | 14 | 1 | 0 | 2 |
| | | | | cell | | | | | | |
| | | | | type | | | | | | |
| 2 | 3 | 9 | 3 | clear | 3 | 17 | 15 | 2 | 0 | 0 |
| | | | | cell | | | | | | |
| | | | | type | | | | | | |
| 8 | 9 | 23 | 11 | Σ | | 51 | 40 | 5 | 2 | 4 |
| 16% | 18% | 45% | 22% | % | | | 78% | 10% | 4% | 8% |
| 2 | 1 | 1 | 1 | Chro- | 1 | 5 | 0 | 1 | 3 | 1 |
| | | | | mo- | | | | | | |
| | | | | pho- | | | | | | |
| | | | | bic | | | | | | |
| 2 | 1 | 4 | 1 | Chro- | 2 | 8 | 0 | 0 | 0 | 8 |
| | | | | mo- | | | | | | |
| | | | | pho- | | | | | | |
| | | | | bic | | | | | | |
| 4 | 2 | 5 | 2 | Σ | | 13 | 0 | 1 | 3 | 9 |
| 31% | 15% | 38% | 15% | % | | | 0% | 8% | 23% | 69% |

In total 64 RCC lesions of different RCC subtypes (clear cell type: 51; chromophobic: 13), histopathologically classified according to Störkel and van der Berg (World J. Urol. 1995, 13, 153), were subjected to immunohistochemical analysis using anti-CK 8 and anti-vimentin mAbs. The results are summarized using the scoring system described in the examples. (+++=strong, ++=intermediate, +=weak and −=very weak or no positive staining)

A strong or intermediate positive CK 8 staining of the cell membranes was detected in 16% and 18% of the RCC lesions and a weak expression of CK 8 was demonstrated in 45% of the tumors analyzed (FIG. 4). A distinct frequency of CK 8 and vimentin expression was found in clear cell and chromophobic RCC (Table 1). 78% and 10% of clear cell RCCs exhibit a strong or intermediate positive cytoplasmic vimentin staining, respectively. A weak vimentin expression was found in 4% of RCCs of this subtype. In contrast, RCC of the chromophobic subtype showed a strong or intermediate positive staining for CK 8 in 31% and 15% of lesions analyzed, whereas a weak CK 8 expression was detectable in 38% of this RCC subtype. Only 8% and 23% of chromophobic RCCs demonstrated an intermediate or weak positive cytoplasmic staining for vimentin, respectively (FIG. 4; Table 1). The observed coexpression of CK 8 and vimentin appear to frequently occur in RCC, especially of the clear cell subtype. Therefore, a combined expression of both proteins may serve as diagnostic marker for the detection of clear cell RCC.

The straining of RCC lesions and normal kidney tissues exhibit a variable stathmin expression pattern (Table 2). While the anti-stathmin antibody stained less than 10% of the epithelium of the proximal and distal tubule system, endothelial cells, inflammatory cells and epithelial cells of compressed peritumoral tubules showed a strong positive cytoplasmic staining. In contrast, tumor cells of the clear cell RCC showed only intermediate or weak positive staining for stathmin in 10% and 33%, respectively, whereas 57% of this RCC subtype totally lack stathmin expression (FIG. 4; Table 2). RCCs of chromophobic subtype exhibit a weak positive straining for stathmin in 60% of lesions analyzed, whereas the other 40% were negative for stathmin staining (FIG. 4; Table 2;).

TABLE 2
Stathmin expression in RCC subtypes

| Stathmin | | | | | | |
|---|---|---|---|---|---|---|
| +++ | ++ | + | − | RCC subtype | G | n |
| 0 | 1 | 3 | 3 | Clear cell type | 1 | 7 |
| 0 | 1 | 1 | 5 | Clear cell type | 2 | 7 |
| 0 | 0 | 3 | 4 | Clear cell type | 3 | 7 |
| 0 | 2 | 7 | 12 | Σ | | 21 |
| 0% | 10% | 33% | 57% | % | | |
| 0 | 0 | 3 | 2 | Chromophobic | 1 | 5 |
| 0 | 0 | 3 | 2 | Chromophobic | 2 | 5 |
| 0 | 0 | 6 | 4 | Σ | | 10 |
| 0% | 0% | 60% | 40% | % | | |

In total 31 RCC lesions of distinct subtype and grading were analyzed by immunohistochemistry using the anti-stathmin mAb. Quantitative analysis was performed according to the scoring system described in the examples. (+++=strong, ++=intermediate, +=weak and −=very weak or no positive staining)

These results show that coexpression of CK 8, vimentin and/or stathmin can be used as diagnostic markers for RCC subtypes.

Therefore the present invention provides methods for the identification of RCC and the differentiation of subtypes of RCC by immunohistochemical staining of tissue samples of kidney epithelium with anti-CK 8, anti-vimentin and/or anti-stathmin antibodies.

In principle the method comprise the following steps:

a) incubating a tissue sample from kidney epithelium obtainable by a individual which is suspected of having RCC with at least one antibody selected from the group consisting of cytokeratin 8, anti-vimentin and anti-stathmin (first antibody) under conditions which ensure the binding of said antibody to the tissue sample and

b) contacting the first antibody with a second antibody comprising a recognition site with binding affinity to the first antibody and a detectable label as described above under conditions which ensure the binding of the antibody to the first antibody,

c) performing a detection step to detect the second antibody bound to the first antibody,

d) comparing the tissue samples detected by step c) with reference samples which have been treated according to steps a) to c) obtained from individuals suffering from the clear cell, chromophobic, chromophilic or the oncocytomic subtype of RCC. The determination of the subtype of RCC for the reference samples can be performed for example as described by Thoenes et al, (Path. Res. Pract. 1986, 181, 125) and Störkel and van der Berg (World J. Urol. 1995, 13, 153).

The present invention provides furthermore a kit containing components for the identification of RCC and the differentiation of subtypes of RCC with immunohistochemical methods.

These may be at least:

a) anti-CK 8, anti-vimentin and/or anti-stathmin antibodies

b) a second antibody bearing a detectable label directed against the first antibody.

### EXAMPLES

### Example 1

Cell Culture and IFN-γ Treatment

MZ1257RC and MZ1940RC represent well defined human cell lines characterized as renal cell carcinoma (RCC) of clear cell type (Seliger, B. et al., Cancer Res. 1996, 56, 1756-60), whereas MZ2733RC and MZ2733NN its corresponding normal renal tissue were recently established from a patient with primary RCC of clear cell type. All RCC lines were maintained in DMEM supplemented with 10% fetal calf serum, 2 mM glutamine and 100 U/ml penicillin/100 μg/ml streptomycin).

IFN-γ-treatment of MZ1257RC cells was performed for 48 h in the presence of 300 U/ml recombinant IFN-γ (Imukin, Boehringer Ingelheim, Ingelheim, Germany).

Serum Samples:

All serum samples were isolated from venous human blood samples taken from either patients diagnosed with renal cell carcinoma or from normal volunteer donors after informed consent was given from each individual.

### Example 2

Two-Dimensional Gel Electrophoresis

Sample Preparation:

The cell lines were expanded to cell counts of 5×10⁷ to 1×10⁸ cells per batch and then harvested by trypsination. The cell pellets were washed 3-4 times in phosphate buffered saline (PBS) and thereafter stored in sterile cryotubes as dry cell pellets in aliquots of 5×10⁶ or 1×10⁷ cells/tube in liquid nitrogen until further use. Cell pellets were resuspended in lysis buffer (7M urea, 2M thiourea, 0.2M dimethyl-benzylammonium propane sulfonate (NDSB), 1% dithiothreitol (DTT), 4% 3-[(3-cholamidopropyl)dimethyl-ammino]-1-propane-sulfonate (CHAPS), 0.5% pharmalytes and a trace of the dye bromophenol blue. The lysate was sonicated (3×4 min in a ultra sonicator bath) and then cleared by centrifugation in a micro centrifuge (90 min, 15° C., 13.000 rpm).

Protein Quantitation:

Protein quantitation was performed according to a protocol described by Ramagli and Rodriguez which allows the use of the original Bradford method even in the presence of high amounts of urea. Briefly replicates of 2.5 μl-10 μl aliquots of the cleared lysate were adjusted to a final volume of 10 μl and each sample mixed with 10 μl 0.1M HCl. Subsequently 80 μl ddH₂O were added to each sample and the samples then mixed again. To each replicate sample (100 μl) 3.5 ml of 1:3 diluted dye reagent mix (Bio-Rad Protein Assay Dye Reagent Concentrate) was added and the mixture blended by gentle vortexing. After 5 minutes, absorbance at 595 nm was measured in plastic cuvettes using an reagent blank (10 μl lysis buffer, processed as described above) as a reference.

Sample Loading/Isoelectric Focussing and Strip Equilibration:

Lysates were adjusted with fresh lysis buffer to a final volume of 350 μl each, from which 340 μl were transferred into IPGphor strip holders (Amersham Pharmacia Biotech). Immobiline DryStrip (pH 3-10, NL, 18 cm, Amersham Pharmacia Biotech) rehydratization and sample loading were performed in a single step. 90 minutes after adding the DryStrips to the lysates the sample soaked strips were covered with 400 μl Immobiline DryStrip Cover Fluid. Isoelectric focussing was performed on a IPGphor unit (Amersham Pharmacia Biotech) at 20° C. using the following parameters: rehydration for 2 h at 0 V; 10 h at 30 V; 1 h at 500 V; 1 h at 1000 V, 1 h at 5000 V, 4-5 h at 8000 V, adding up to 36.000-38.000 Vhrs if the targeted proteins (low molecular weight components) were separated in the second dimension on 16% T/2.5% C SDS-PAGE gels (last step 4h at 8000V)- or 44.000-46.000 Vhrs if the sample lysate was separated on 7% T/2.5% C SDS-PAGE gels targeting high molecular weight components (last step 5h at 8000V). All steps were run in the step and hold mode. Focused strips were either stored at −80° C. or directly subjected to the strip equilibration procedure, which was performed by incubating the strips for 15 minutes in 10 ml equilibration buffer (50 mM Tris-HCl pH 8.8, 6M urea, 30% glycerol, 2% SDS) supplemented with 1.5% DTT followed by 15 minutes incubation in 10 ml equilibration buffer supplemented with 4.8% jodacetamide.

Second-Dimension SDS-PAGE:

SDS-PAGE separation was performed using a Hoefer ISO-DALT System (Amersham Pharmacia Biotech) and run in polyacrylamid/piperazine diacrylamide (PDA) PAGE gels. The gel mix contained 375 mM Tris/HCl, pH 8.8, 5 mM Na₂S₂O₄, and 4% glycerol but no sodium dodecylsulfate (SDS). Freshly equilibrated Immobiline DryStrips were transferred onto the surface of thoroughly rinsed PAGE gels. Strip immobilization was achieved by embedding the strips in 1% soft melting agarose containing traces of marker dyes (bromophenol blue for 7% T/2.5% C gels; bromophenol blue plus xylene cyanole FF for 16% T/2.5% C gels). Gels were run in SDS-PAGE running buffer (25 mM Tris, 192 mM glycine, 0.1% SDS) under strict temperature control (<20° C.) until the dye front reached the end of the gel (16% T/2.5% C gels were run until the xylene cyanole FF dye front eluted from the gel). The initial transfer of the sample form the isoelectric focussing (IEF) strip into the gel was performed at low voltage (1 h at constant 50 V), whereas the separation was run at constant high voltage (100-140 V).

Gel Staining:

Gels were stained with colloidal Coomassie Blue. All gels were scanned on a conventional scanner (Hewlett Packard ScanJet 6100C) at a resolution of 600 dpi, and stored as TIFF-images.

Gels destined for Western-Blot analyses or gels containing protein spots which were subjected for mass spectrometry analyses were merely stained with colloidal Coomassie Blue staining solution (10% ammonium sulfate, 2% phosphoric acid, 0.1% Coomassie Brilliant Blue G-250, 20% methanol, thus skipping the initial fixation step and thereafter destained by extensive washing in H₂O (dd).

### Example 3

Immunoblotting

For immunoblot analyses, colloidal Coomassie Blue prestained 2-D PAGE gels were blotted onto Immobilon P membranes with the ISO-DALT tank blotting system (Amersham Pharmacia Biotech) using SDS-PAGE running buffer supplemented with 20% methanol as transfer buffer and applying 500 Vhrs per transfer. Blots were subsequently incubated for 1 h in blocking solution (140 mM NaCl, 10 mM Tris/HCl pH 7.4, 0.4% Tween 20, 5% low fat dry milk and 10% horse serum, rinsed twice in Tris-buffered saline (TBS; 140 mM NaCl, 10 mM Tris/HCl pH 7.4) and then incubated over night at 4° C. with either control or patient sera (20 ml/membrane) diluted 1:20 in antibody incubation buffer (TBS, 0.1% Tween 20, 2% low fat dry milk). Then, the membranes were washed 3 times (10 minutes each) in TBS, 0.4% Tween 20 and incubated at room temperature for 0.5-1 h with a horseradish peroxidase (HRP)-conjugated secondary mAb solution (20 ml/membrane, rabbit anti-human IgG, diluted 1:1000 in antibody incubation buffer). Following 3 washing steps with TBS, 0.4% Tween 20 spot visualization was carried out with a chemiluminescence detection kit (Lumi-Light Western Blotting Substrate, Roche Molecular Biochemicals, Mannheim) according to the manufacturer's instructions and recorded on scientific imaging film (Kodak X-Omat Blue XB-1). Signal to spot matching was per-formed by superimposing imaging films and corresponding gel prints.

### Example 4

Mass Spectrometry

For mass spectrometry immunostained protein spots were excised from a colloidal Coomassie Blue-stained duplicate gel. Each sample was transferred into a sterile micro reaction tube, the gel slices incubated for 30 minutes in 50 mM NH₄HCO₃/acetonitril (60%/40%) at 30° C. and the resulting supernatants subsequently removed and discarded. Gel slices were then vacuum dried and stored at −80° C. until further use. For in-gel digestion, each sample was soaked for 1 h in 25-40 pl of 50 mM NH₄HCO₃ containing 0.1 μg/ml modified trypsin (Promega, Madison, Wis., USA). The supernatants were collected, aliquots of 25 μl fresh NH₄HCO₃ were added and the samples then incubated overnight at 37° C. Peptide extraction was achieved by incubating samples twice for 20 minutes in extraction buffer (H₂O/trifluoroacetic acid (TFA); 50%/50%; v/v) and then twice for 20 minutes in a buffer containing acetonitril/TFA; 50%/50%; v/v). Supernatants from each sample were concentrated to a final volume of about 25-50 μl/sample and then desalted with ZipTips (Millipore) according to the manufactures protocol. One pl aliquots of the resulting eluats were loaded onto the MALDI matrix and directly subjected to peptide mass fingerprinting analyses using a Perseptive Biosystems Voyager RP-DE instrument (Perseptive Biosystems, Framington, Mass.).

### Example 5

Patients and Tissue Samples Used for Immunohistochemistry

For immunohistochemical analysis, surgically removed tissue samples from RCC and corresponding normal kidney epithelium were randomly obtained from patients who had undergone radical nephrectomy. Histopathological classification of each tumor was performed according to the criteria proposed by Thoenes and coworkers (Thoenes et al., Path. Res. Pract. 1986, 181, 125; Störkel and van der Berg, World J. Urol. 1995, 13, 153). These data include gender, stage of disease, tumor invasion, and lymph node involvement according to the TNM (Tumor Node Metastasis) system. In total, 64 primary renal tumors, including 51 clear cell is carcinoma and 13 chromophobic carcinomas as well as 64 autologous kidney specimens were collected at resection. The tissue samples were formalin-fixed and paraffin-embedded.

Immunohistochemistry

Immunohistochemical stainings were performed with the mAbs anti-human cytokeratin 8 (clone βH11, DAKO, Hamburg, Germany, dilution 1:25), anti-vimentin mAb (clone V9, DAKO, dilution 1:40) and anti-stathmin (B37545, Calbiochem, USA, dilution 1:500). For antigen retrieval, consecutive sections were incubated for 8 and 6 minutes in citrate buffer in a microwave oven, respectively, followed by a washing procedure with Tris-buffered saline and an additional incubation with normal swine serum (dilution 1:10) for 10 minutes. Slides were incubated with the primary antibodies for one hour at room temperature. Detection was performed by using the LASB (Labeled Streptavidin Biotin)-peroxidase kit and AEC (Amino-9-ethylcarbazole) as described (DAKO Diagnostika GmbH, Hamburg, Germany). Negative controls where performed by omitting the primary antibody.

Quantitative analysis for each tumor was performed according to the following score:

| % positive tumor cells/ | | |
|---|---|---|
| specimen | Score points | Classification |
| <5 | − | Negative |
| >5 and <25 | + | Weak positive |
| >26 and <50 | ++ | Intermediate |
| | | positive |
| >50 | +++ | Strong positive |

1) use of at least one protein selected from the group consisting of β-actin, γ-actin, α-tubulin, cytokeratin, cytokeratin 8, cytoskeletal tropomyosin, f-actin capping protein, hsp 27, hsp 60, hsp 70, hsp 90, grp 78 (BIP), gp 96, gluthathione-S-transferase, gluthathione synthetase, superoxide dismutae, thioredoxin peroxidase, PA28α, ubiquitin thiolesterase, triosephosphate isomerase, aldose reductase, enoyl-CoA hydratase, α-enolase, annexin II, IV and V, stathmin, nicotinamide-N-methyltransferase, B23/nucleophosmin and vimentin as tumor marker.

2) Use of a protein as defined in claim 1 and/or β-tubulin as tumor marker for renal cell carcinoma.

3) A in-vitro method for diagnosis and prognosis of cancer in an individual, comprising detecting by means of an immunoassay the presence of an antibody obtained from a serum sample of said individual and directed to a tumor marker protein as defined in claim 1 or 2 which is present in the serum.

4) The method of claim 3 wherein the immunoassay comprises the following steps:
(a) immobilizing a protein according to claim 1 onto a membrane or substrate,
(b) contacting the membrane or substrate with a serum sample of an individual and
(c) detecting the presence of tumor marker-specific antibodies in the serum sample of the individual.

5) The method of claim 4 wherein the tumor marker specific antibody in the serum sample is detected by means of an exogenously applied labeled antibody, directed to said serum antibody.

6) The method according to any of the claims 3 to 5 wherein the individual is suffering from renal cell carcinoma.

7) A diagnostic kit suitable for performing the method of any of the claims 3 to 6 comprising at least one or more of the tumor markers as defined in claim 1 or 2.

8) The use of at least one tumor marker as defined in claim 1 or 2 for the manufacture of a medicament for stimulating an immune response in a individual.

9) The use of at least one antibody or a fragment thereof which immunospecifically binds to at least one of the tumor markers as defined in claim 1 or 2 for the manufacture of a medicament to elicit a reaction which facilitates killing of tumor cells and/or inhibiting tumor cell growth.

10) The use of claim 9, wherein the antibody or the fragment thereof is an antibody fusion protein.

11) A pharmaceutical composition comprising at least one tumor marker as defined in claim 1 or 2 and optionally an pharmaceutically acceptable carrier, diluent or excipient.

12) A pharmaceutical composition comprising at least one antibody or fragments thereof which immunospecifically bind to at least one of the tumor markers as defined in claim 1 or 2 and optionally an pharmaceutically acceptable carrier, diluent or excipient.

13) A pharmaceutical composition of claim 12 wherein the antibody or the fragment thereof is an antibody fusion protein.

14) A pharmaceutical composition according to any of the claims 11 to 13 comprising additionally a chemotherapeutic agent.

15) A pharmaceutical package comprising in a first container a pharmaceutical composition according to any of the claims 11 to 13 and in a second container a pharmaceutical composition comprising a chemotherapeutic agent for simultaneous or timely shifted administration.

16) Use of at least one antibody selected from the group consisting of anti-cytokeratin 8, anti-vimentin and anti-stathmin for the identification of RCC and the differentiation of subtypes of RCC with immunohistochemical methods.

17) A method for the identification of RCC and the differentiation of subtypes of RCC comprising the steps
a) incubating a tissue sample from kidney epithelium obtainable by a individual which is suspected of having RCC with at least one antibody selected from the group consisting of anti-cytokeratin 8, anti-vimentin and anti-stathmin (first antibody) under conditions which ensure the binding of said antibody to the tissue sample and
b) contacting the first antibody with a second antibody comprising a recognition site with binding affinity to the first antibody and a detectable label under conditions which ensure the binding of the antibody to the first antibody,
c) performing a detection step to detect the second antibody bound to the first antibody,
d) comparing the tissue samples detected by step c) with reference samples obtained from individuals suffering from the clear cell, chromophobic, chromophilic or the oncocytomic subtype of RCC.

18) A kit for the identification of RCC and the differentiation of subtypes of RCC comprising
a) at least one antibody according to claim 16 (first antibody)
b) at least one second antibody bearing a detectable label directed against the first antibody
